(19) United States
(12) Patent Application Publication
(10) Pub. No.: US 2026/0253982 A1
(43) Pub. Date: Aug. 27, 2026

(54) METHOD FOR RESTORING SECONDARY BATTERY

(71) Applicant: SK ON CO., LTD., Seoul (KR)

(72) Inventors: Seong Yun LEE, Daejeon (KR); Sang Mo KIM, Daejeon (KR)

(21) Appl. No.: 19/545,025

(22) Filed: Feb. 20, 2026

(30) Foreign Application Priority Data
Feb. 24, 2025 (KR) 10-2025-0023803

(51) Int. Cl. H01M 10/52; H01M 50/103

(52) U.S. Cl. CPC H01M 10/52; H01M 50/103

(57) ABSTRACT

Proposed is a method for restoring a secondary battery, the method may include an appearance inspection step of inspecting the appearance of a secondary battery, a sorting step of sorting out a defective product detected in the appearance inspection step, and a restoration step of restoring a defective portion of the defective product sorted out in the sorting step using a restoration device among a plurality of restoration devices corresponding to the defective portion.

### CROSS REFERENCE TO RELATED APPLICATION

The present application claims priority to Korean Patent Application No. 10-2025-0023803, filed February 24, 2025, the entire contents of which is incorporated herein for all purposes by this reference.

### BACKGROUND

### Technical Field

The present disclosure relates to a method for restoring a secondary battery.

### Description of the Related Art

In general, a secondary battery is a reusable battery that converts chemical energy into electrical energy during discharge and then can be recharged by converting electrical energy back into chemical energy.

Secondary batteries may include nickel-cadmium (Ni-Cd) batteries, nickel-hydrogen (Ni-MH) batteries, lithium-metal batteries, lithium-ion (Li-Ion) batteries, and lithium-ion polymer batteries.

Among secondary batteries, lithium secondary batteries have an expected lifespan of around 500 cycles and a short charging time of about 1 to 2 hours. Lithium secondary batteries are about 30 to 40% lighter than nickel-hydrogen batteries, allowing for weight reduction. Lithium secondary batteries generally have the highest voltage per unit cell among commonly used secondary batteries, and possess excellent energy density, making them ideal for mobile devices.

### SUMMARY

According to an aspect of the present disclosure, provided is a method for restoring a secondary battery, the method being capable of restoring appearance defects.

According to another aspect of the present disclosure, provided is a method for restoring a secondary battery, which can be widely applied in green technology fields, such as electric vehicles, battery charging stations, and renewable energy systems utilizing batteries, including solar and wind power generation.

A method for restoring a secondary battery according to an embodiment of the present disclosure may include: an appearance inspection step of inspecting the appearance of a secondary battery; a sorting step of sorting out a defective product detected in the appearance inspection step; and a restoration step of restoring a defective portion of the defective product sorted out in the sorting step using a restoration device among a plurality of restoration devices corresponding to the defective portion.

The restoration step may include a bubble removal step of removing bubbles formed in the defective portion using a bubble removal device among the restoration devices.

The bubble removal step may involve moving the defective product to the bubble removal device and positioning the defective portion to face the bubble removal device.

The bubble removal step may involve perforating the defective portion to form micro-holes by means of the bubble removal device.

The restoration step may include a damage recovery step of moving the defective product to a damage restoration device among the restoration devices.

The damage recovery step may involve positioning the defective portion to face the damage restoration device.

The damage recovery step may involve irradiating the defective portion with plasma by means of the damage restoration device and then performing heat pressing.

The restoration step may include a roughness recovery step of moving the defective product to a roughness restoration device among the restoration devices, and positioning the defective portion of the defective product to face the roughness restoration device.

The roughness recovery step may involve etching an area around the defective portion of the defective product by means of the roughness restoration device.

A method for restoring a secondary battery according to another embodiment of the present disclosure may include: an appearance inspection step of inspecting the appearance of a secondary battery; a sorting step of sorting out a defective product detected in the appearance inspection step; and a restoration step of restoring a defective portion of the defective product sorted out in the sorting step using a restoration device among a plurality of restoration devices corresponding to the defective portion, wherein the restoration step may include a damage recovery step of moving the defective product to a damage restoration device among the restoration devices, wherein the damage recovery step may involve irradiating the defective portion of the defective product with plasma.

The damage recovery step may involve heat-pressing the defective portion of the defective product by means of the damage restoration device.

The restoration step may include a roughness recovery step of moving the defective product to a roughness restoration device among the restoration devices, and positioning the defective portion of the defective product to face the roughness restoration device.

The roughness recovery step may involve etching an area around the defective portion of the defective product by means of the roughness restoration device.

The features and advantages of the present disclosure will become more apparent from the following detailed description based on the accompanying drawings.

Prior to this, terms or words used in this specification and claims should not be construed in their usual, dictionary meaning, and should be interpreted with meaning and concept consistent with the technical idea of the present disclosure on the basis of the principle that the inventor can define terminology appropriately to explain his or her invention in the best way possible.

According to an embodiment of the present disclosure, it is possible to reduce the quantity of defective products to be discarded and thereby improve productivity.

In addition, the present disclosure can restore the appearance of a defective product without replacing materials, thereby reducing material costs.

In addition, the present disclosure can replace reworking using adhesive members such as tape, thereby improving product quality.

In addition, the present disclosure can be widely applied to prismatic batteries, pouch-type batteries, and the like.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a flowchart showing a method for restoring a secondary battery according to an embodiment of the present disclosure;

FIG. 2 is a view showing a bubble removal step in a method for restoring a secondary battery according to an embodiment of the present disclosure;

FIG. 3 is a view showing a damage recovery step in a method for restoring a secondary battery according to an embodiment of the present disclosure; and

FIG. 4 is a view showing a roughness recovery step in a method for restoring a secondary battery according to an embodiment of the present disclosure.

### DETAILED DESCRIPTION

Terms used to describe an embodiment of the present disclosure are not intended to limit the disclosure. It should be noted that singular expressions include plural expressions unless the context clearly dictates otherwise.

It should be noted that, in assigning reference numerals to components in the drawings, identical components are assigned the same reference numerals as much as possible even if they are shown in different drawings, and similar reference numbers are assigned to similar components.

The drawings may be schematic or exaggerated for the purpose of illustrating the embodiments. In this document, expressions such as “have”, “may have”, “include”, or “may include” refer to the presence of the corresponding feature (e.g., a numerical value, function, operation, or component such as a part), and do not exclude the presence of additional features.

Terms such as “one”, “other”, “another”, “first”, “second”, etc., are used to distinguish one component from another component, and the components are not limited by the terms.

Hereinafter, an embodiment of the present disclosure will be described in detail with reference to the attached drawings.

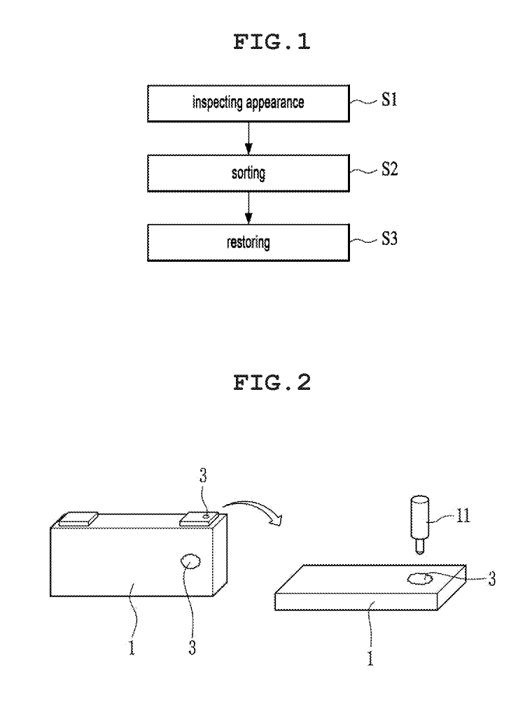

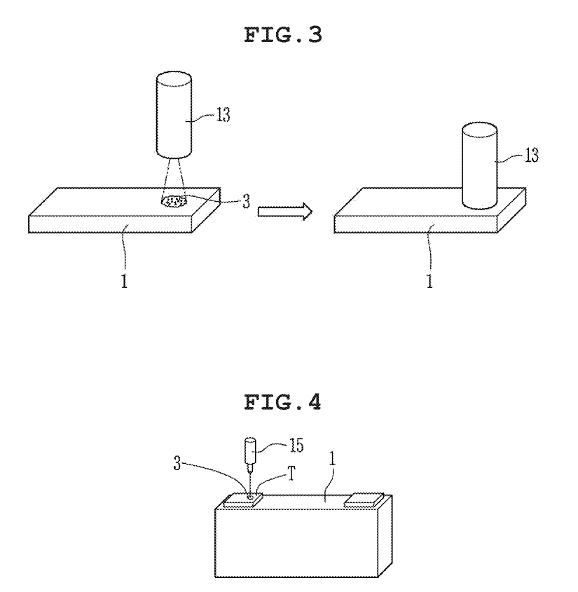

FIG. 1 is a flowchart showing a method for restoring a secondary battery according to an embodiment of the present disclosure; FIG. 2 is a view showing a bubble removal step in a method for restoring a secondary battery according to an embodiment of the present disclosure; FIG. 3 is a view showing a damage recovery step in a method for restoring a secondary battery according to an embodiment of the present disclosure; and FIG. 4 is a view showing a roughness recovery step in a method for restoring a secondary battery according to an embodiment of the present disclosure.

Referring to FIG. 1, a method for restoring a secondary battery according to an embodiment of the present disclosure may include: an appearance inspection step S1 of inspecting the appearance of a secondary battery 1; a sorting step S2 of sorting out a defective product detected in the appearance inspection step S1; and a restoration step S3 of restoring a defective portion 3 of the defective product sorted out in the sorting step S2 using a restoration device 11, 13, or 15 corresponding to the defective portion 3.

In the method for restoring a secondary battery according to an embodiment of the present disclosure, the secondary battery 1 may be a prismatic secondary battery or a pouch-type secondary battery.

The appearance inspection step S1 may involve inspecting appearance defects in the secondary battery 1.

The appearance defects may include bubbles, scratches, dents, impressions, or punctures occurring in a polymer layer including adhesive members such as tape or film attached to the appearance of the secondary battery 1 and a body such as a pouch, as well as scratches, dents, impressions, folds, or bends occurring on tabs or terminals T.

The secondary battery 1 in which such appearance defects are detected may be classified as a defective product even if the defects do not affect the performance of the secondary battery 1.

The secondary battery 1 with detected appearance defects may be converted into a good product by restoring the defects, thus improving productivity and reducing manufacturing costs.

In the appearance inspection step S1, in case that appearance defects are detected in the secondary battery 1, data regarding the unique serial number of the secondary battery 1 and the defective portion 3 may be recorded in a controller (not shown).

The sorting step S2 may involve sorting and storing defective products detected in the appearance inspection step (S1) and good products in which no appearance defects are detected.

The restoration step S3 may involve restoring defective portions 3 of defective products stored in the sorting step S2 using a restoration device 11, 13, or 15 corresponding each defective portion 3, thereby restoring the secondary batteries as good products without discarding or reworking the defective products.

In the restoration step S3, the defective portions 3 corresponding to the unique serial numbers of defective products sorted and stored in the sorting step S2 may be identified using data recorded in the controller.

In addition, in the restoration step S3, the defective portions 3 corresponding to the unique serial numbers of the secondary batteries 1 may be restored using the restoration devices 11, 13, and 15 corresponding to the defective portions 3.

As a first embodiment, the restoration device may include a bubble removal device 11 for removing bubbles occurring in a polymer layer including adhesive members such as tape or film and a body such as a pouch.

As the first embodiment, the restoration step S3 may include a bubble removal step.

The bubble removal step may involve removing bubbles occurring in a defective portion 3 of a defective product using the bubble removal device 11 among the restoration devices 11, 13, and 15.

The bubble removal device 11 may be a device such as a micro-needle capable of perforating fine micro-holes in the polymer layer.

Referring to FIG. 2, in the bubble removal step, a defective product may be moved to the bubble removal device such that a bubble-defect portion of a defective portion 3 faces the bubble removal device.

In addition, in the bubble removal step, the bubble removal device may perforate micro-holes 3a in the bubble-defect portion of the defective portion 3 of the defective product.

The bubbles formed in the defective product may be discharged to the outside through the perforated micro-holes.

As a second embodiment, the restoration device may include a damage restoration device 13 for restoring scratches, dents, impressions, punctures, and the like occurring in a polymer layer including adhesive members such as tape or film and a body such as a pouch, as well as folds, bending, and the like occurring in tabs or terminals T.

As the second embodiment, the restoration step S3 may include a damage recovery step.

In the damage recovery step, a defective product may be moved to the damage restoration device 13 among the restoration devices 11, 13, and 15.

In addition, in the damage recovery step, a defective portion 3 of the defective product may be positioned to face the damage restoration device 13.

Referring to FIG. 3, in the damage recovery step, the defective portion 3 of the defective product may be irradiated with plasma by the damage restoration device 13 and subsequently heated and pressed using a heat press or the like.

In addition, in the restoration step S3, after the bubble removal step of the first embodiment, the micro-holes 3a perforated in the defective product may be removed in the damage recovery step of the second embodiment using the damage restoration device 13, thereby restoring the defective product to a good secondary battery.

As a third embodiment, the restoration step S3 may include a roughness recovery (reversing surface degradation) step in which, upon the occurrence of surface roughness variations due to scratches, dents, or impressions in tabs or terminals T, the average roughness of the tabs or terminals T is recovered using the roughness restoration device 15.

As an embodiment, the average roughness of a positive electrode tab may range from 0.210 µm to 0.221 µm.

The average roughness of a negative electrode tab may range from 0.510 µm to 0.845 µm.

The average roughness of a terminal T may range from 0.200 µm to 0.220 µm.

However, in the method for restoring a secondary battery according to the present disclosure, the average roughness values of the tabs and terminals T are not limited to the aforementioned values, and predetermined average roughness values optimized for a product may be established.

In the roughness recovery step, a defective product may be moved to the roughness restoration device 15 among the restoration devices.

In addition, in the roughness recovery step, a defective portion 3 of the defective product may be positioned to face the roughness restoration device 15.

Referring to FIG. 4, in the roughness restoration step, the defective portion 3 of the defective product may be etched using the roughness restoration device 15 to restore the average roughness of the tabs or terminals T.

The roughness restoration device may be a device that etches by any one of sputtering etching, ion beam etching, plasma etching, or reactive ion etching.

According to the method for restoring a secondary battery according to an embodiment of the present disclosure described above, in the case of a prismatic secondary battery, the restoration step S3 corresponding to the defective portions 3 of the defective product may be executed by any one or more of the first, second, and third embodiments described above, thereby restoring the defective product to a good product.

According to the method for restoring a secondary battery according to an embodiment of the present disclosure described above, in the case of a pouch-type secondary battery, the restoration step S3 corresponding to the defective portions 3 of the defective product may be executed by any one or more of the second and third embodiments described above, thereby restoring the defective product to a good product.

A method for restoring a secondary battery according to another embodiment of the present disclosure may include: an appearance inspection step S1 of inspecting the appearance of a secondary battery 1; a sorting step S2 of sorting out a defective product detected in the appearance inspection step S1; and a restoration step S3 of restoring a defective portion 3 of the defective product sorted out in the sorting step S2 using a restoration device 11, 13, or 15 corresponding to the defective portion 3. The restoration step S3 may include a damage recovery step of moving the defective product to the damage restoration device 13 among the restoration devices 11, 13, and 15. The damage recovery step may involve irradiating the defective portion 3 of the defective product with plasma.

In the damage recovery step, the defective portion 3 of the defective product may be heat-pressed using the damage restoration device 13, such as a heat press.

In addition, the restoration step S3 may further include a roughness recovery step in which the defective product is transferred to the roughness restoration device 15 among the restoration devices 11, 13, and 15, and the defective portion 3 of the defective product is aligned to face the roughness restoration device 15.

In the roughness recovery step, the area surrounding the defective portion 3 of the defective product may be etched using the roughness restoration device 15.

As described above, the method for restoring a secondary battery according to another embodiment of the present disclosure may be a restoration method for a pouch-type secondary battery.

That is, the damage restoration device 13 may restore the defective portion 3 resulting from scratches, dents, depressions, perforations, or the like occurring in the polymer layer including the body such as the pouch of the defective product, and from folds or bends occurring in the tabs or terminals T, by applying plasma onto the defective portion 3 and then performing heat pressing.

Subsequently, the roughness restoration device 15 may restore roughness deformations caused by scratches, dents, depressions, or the like occurring in the defective portion 3 of the tabs or terminals T through etching.

Above, the present disclosure has been described in detail through specific embodiments. The embodiments are for specifically explaining the present disclosure, and are only illustrative and do not limit the scope of the appended claims. It is obvious to those skilled in the art that various changes and modifications to the embodiments are possible within the scope and technical idea of the present disclosure, and it is natural that such changes and modifications fall within the scope of the appended claims.

What is claimed is:

1. A method for restoring a secondary battery, the method comprising:
an appearance inspection step of inspecting an appearance of a secondary battery;
a sorting step of sorting out a defective product detected in the appearance inspection step; and
a restoration step of restoring a defective portion of the defective product sorted out in the sorting step using a restoration device among a plurality of restoration devices corresponding to the defective portion.

2. The method of claim 1, wherein the restoration step comprises:
a bubble removal step of removing bubbles formed in the defective portion using a bubble removal device among the restoration devices.

3. The method of claim 2, wherein the bubble removal step involves moving the defective product to the bubble removal device and positioning the defective portion to face the bubble removal device.

4. The method of claim 3, wherein the bubble removal step involves perforating the defective portion to form micro-holes by means of the bubble removal device.

5. The method of claim 4, wherein the restoration step comprises:
a damage recovery step of moving the defective product to a damage restoration device among the restoration devices.

6. The method of claim 5, wherein the damage recovery step involves positioning the defective portion to face the damage restoration device.

7. The method of claim 6, wherein the damage recovery step involves irradiating the defective portion with plasma by means of the damage restoration device and then performing heat pressing.

8. The method of claim 1, wherein the restoration step comprises:
a roughness recovery step of moving the defective product to a roughness restoration device among the restoration devices, and positioning the defective portion of the defective product to face the roughness restoration device.

9. The method of claim 8, wherein the roughness recovery step involves etching an area around the defective portion of the defective product by means of the roughness restoration device.

10. A method for restoring a secondary battery, the method comprising:
an appearance inspection step of inspecting an appearance of a secondary battery;
a sorting step of sorting out a defective product detected in the appearance inspection step; and
a restoration step of restoring a defective portion of the defective product sorted out in the sorting step using a restoration device among a plurality of restoration devices corresponding to the defective portion,
wherein the restoration step comprises:
a damage recovery step of moving the defective product to a damage restoration device among the restoration devices,
wherein the damage recovery step involves irradiating the defective portion of the defective product with plasma.

11. The method of claim 10, wherein the damage recovery step involves heat-pressing the defective portion of the defective product by means of the damage restoration device.

12. The method of claim 11, wherein the restoration step comprises:
a roughness recovery step of moving the defective product to a roughness restoration device among the restoration devices, and positioning the defective portion of the defective product to face the roughness restoration device.

13. The method of claim 12, wherein the roughness recovery step involves etching an area around the defective portion of the defective product by means of the roughness restoration device.
